(19) United States
(12) Patent Application Publication
(10) Pub. No.: US 2018/0228963 A1
(43) Pub. Date: Aug. 16, 2018

(54) SPLASH BLEU

(71) Applicant: Traci Mitchell, Henderson, NV (US)

(72) Inventor: Traci Mitchell, Henderson, NV (US)

(21) Appl. No.: 15/835,417

(22) Filed: Dec. 7, 2017

Related U.S. Application Data
(60) Provisional application No. 62/458,561, filed on Feb. 13, 2017.

(51) Int. Cl. A61M 3/02; B65D 35/24; A45C 13/10; A61K 8/22; A61Q 19/10

(52) U.S. Cl. CPC A61M 3/0262; A61M 3/0279; B65D 35/24; A45C 13/103; A45C 2013/1015; A61K 8/22; A61Q 19/10; A61M 2209/06; A61M 2210/1475; A45C 13/1076

(57) ABSTRACT

A personal hygiene bottle and kit includes a mini squeeze bottle filled with water and H₂O₂ (hydrogen peroxide) solution. This squeeze bottle will be used to splash away the particles and debris on and near the vagina during a woman's menstrual cycle. This product will be sold separately in a dual pack, or in a kit which will include a carrying pouch with dry wipes, two Splash, and a mini-douche with water and H₂O₂ solution. The present invention eliminates particles and bacteria that may occur during a woman's menstruation cycle. The water and H₂O₂ solution (mini douche) kills bacteria.

### CROSS-REFERENCE TO RELATED APPLICATION

The present application is related to and claims priority from prior provisional application Ser. No. 62/458,561, filed Feb. 13, 2017 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. FIELD OF THE INVENTION

The present invention relates generally to the field of personal hygiene systems and more specifically relates to a personal hygiene bottle and kit comprising a cleaning solution formed as a water and H₂O₂ solution structured and arranged to be used to splash away particles and debris on and near the vagina during a woman's menstrual cycle.

### 2. DESCRIPTION OF THE RELATED ART

A douche is a device used to introduce a stream of water into the body for medical or hygienic reasons, or the stream of water itself. Douche usually refers to vaginal irrigation, the rinsing of the vagina, but it can also refer to the rinsing of any body cavity. A douche bag is a piece of equipment for douching—a bag for holding the fluid used in douching. To avoid transferring intestinal bacteria into the vagina, the same bag must not be used for an enema and a vaginal douche.

Douching after sexual intercourse is not an effective form of birth control. Additionally douching is associated with a number of health problems (cervical cancer, pelvic inflammatory disease, endometritis, and increased risk of sexually transmitted infections) and thus is not recommended.

Various attempts have been made to solve problems found in personal hygiene systems art. Among these are found in: U.S. Pat. No. 5,858,010 to Melodye W. Berry; U.S. Publication No. 2010/0258140 to Sharon H. Creed; and U.S. Pat. No. 4,392,492 to Ernest Pick. This prior art is representative of personal hygiene systems that when used may help a woman feel fresh and clean during a menstrual cycle.

Ideally, a personal hygiene bottle and kit should be user-friendly and safe in-use and, yet should operate reliably and be manufactured at a modest expense. Thus, a need exists for a personal hygiene bottle and kit comprising a cleaning solution formed as a water and H₂O₂ solution structured and arranged to be used to splash away particles and debris on and near the vagina during a woman's menstrual cycle and to avoid the above-mentioned problems.

### BRIEF SUMMARY OF THE INVENTION

In view of the foregoing disadvantages inherent in the known personal hygiene systems art, the present invention provides a personal hygiene bottle and kit (entitled Splash Bleu). The general purpose of the present invention, which will be described subsequently in greater detail is to provide a personal hygiene bottle and kit comprising a cleaning solution formed as a water and H₂O₂ solution structured and arranged to be used to splash away particles and debris on and near the vagina during a woman's menstrual cycle.

A personal hygiene bottle comprising: elongated container portion; a flexible wall; an aperture; a first nozzle portion; and a second nozzle portion. The elongated container portion includes an elongated interior volume adapted to retain a cleaning solution therein. The elongated container portion forms an elongated axis; The flexible wall is adapted such that when the flexible wall is squeezed said cleaning solution can be forced outwardly therefrom. The aperture has a center point offset from the axis of the elongated container portion and is adapted to allow the cleaning solution to be forced outwardly from the elongated container portion.

The first nozzle portion includes attachment portion, an elongated neck portion, and a single aperture. The attachment portion adapted to be releasably connected to the elongated container portion and surround the aperture thereof, such that the cleaning solution can be forced into the nozzle portion. The elongated neck portion adapted to allow the cleaning solution to pass therethrough. The single aperture located on a distal end thereof adapted to allow the cleaning solution to flow outwardly therefrom such that the cleaning solution flows in a single stream at an angle to the axis of said elongated container portion.

The second nozzle portion includes an attachment portion, an elongated neck portion, and a plurality of spaced apertures located on a distal end thereof. The attachment portion adapted to be releasably connected to the elongated container portion and surround said aperture thereof, such that the cleaning solution can be forced into the nozzle portion. The elongated neck portion adapted to allow the cleaning solution to pass therethrough.

The plurality of spaced apertures located on a distal end thereof each adapted to allow said cleaning solution to flow outwardly therefrom in a plurality of streams at differing angles to the axis of said elongated container portion. A user can choose between the first and second nozzle portion depending on the desired function of the second bottle member.

A personal hygiene kit comprising: a first bottle member; a second bottle member; a plurality of dry wipe members; a mini-douche member; a cleaning solution; and a carrying pouch member. The second bottle member includes an elongated container portion forming an elongated axis, including an elongated interior volume adapted to retain a cleaning solution therein. Wherein the elongated interior volume of the second bottle member is larger than the interior volume of the container portion of the first bottle member. The cleaning solution is adapted to be placed within the respective interior volumes of the first and second bottle members. The carrying pouch member is adapted to releasably retain the first and second bottle members, the plurality of dry wipe members, the mini-douche member, and the cleaning solution therein.

The present invention holds significant improvements and serves as a personal hygiene bottle and kit. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, personal hygiene bottle and kit (entitled Spash Bleu), constructed and operative according to the teachings of the present invention.

FIG. 1 shows a perspective view illustrating a personal hygiene bottle and cleansing solution according to an embodiment of the present invention.

FIG. 2 shows a perspective view illustrating the personal hygiene bottle in an in-use condition according to an embodiment of the present invention.

FIG. 3 is a perspective view illustrating a second nozzle portion of the personal hygiene bottle according to an embodiment of the present invention.

FIG. 4 is a perspective view illustrating the personal hygiene bottle comprising: elongated container portion; a flexible wall; an aperture; a first nozzle portion; and a second nozzle portion according to an embodiment of the present invention of FIG. 1.

FIG. 5 is a perspective view illustrating a personal hygiene kit according to an embodiment of the present invention of FIG. 1.

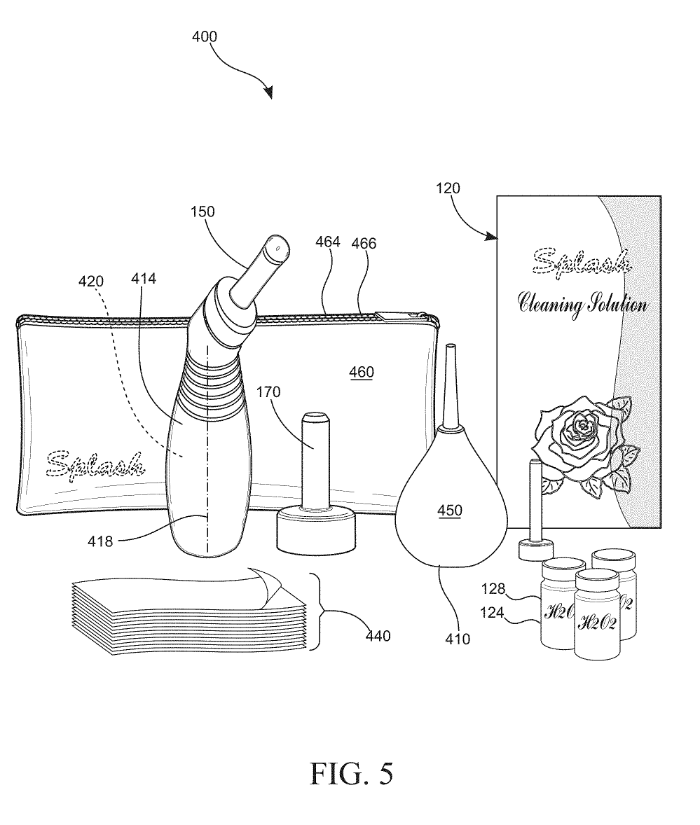

FIG. 6 is a perspective view illustrating a personal hygiene kit according to an embodiment of the present invention of FIG. 1.

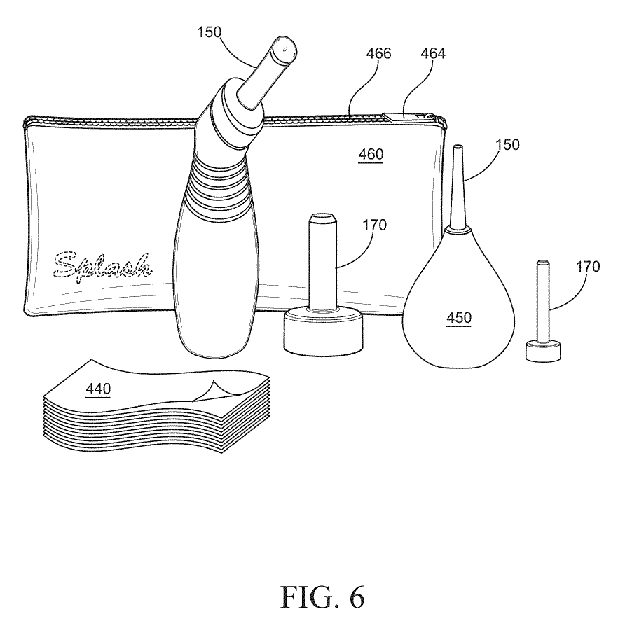

The various embodiments of the present invention will hereinafter be described in conjunction with the appended drawings, wherein like designations denote like elements.

### DETAILED DESCRIPTION

As discussed above, embodiments of the present invention relate to personal hygiene system and more specifically relates to a personal hygiene bottle and kit (entitled Spash Bleu) comprising a cleaning solution formed as a water and H₂O₂ solution structured and arranged to be used to splash away particles and debris on and near the vagina during a woman's menstrual cycle.

Generally speaking, Splash Bleu is a mini squeeze bottle filled with water and H₂O₂ (hydrogen peroxide) solution. This squeeze bottle will be used to splash away the particles and debris on and near the vagina during a woman's menstrual cycle. This product will be sold separately in a dual pack, or in a kit which will include a carrying pouch with dry wipes, two Splash, and a mini-douche with water and H₂O₂ solution. SPLASH eliminates particles and bacteria that may occur during a woman's menstruation cycle.

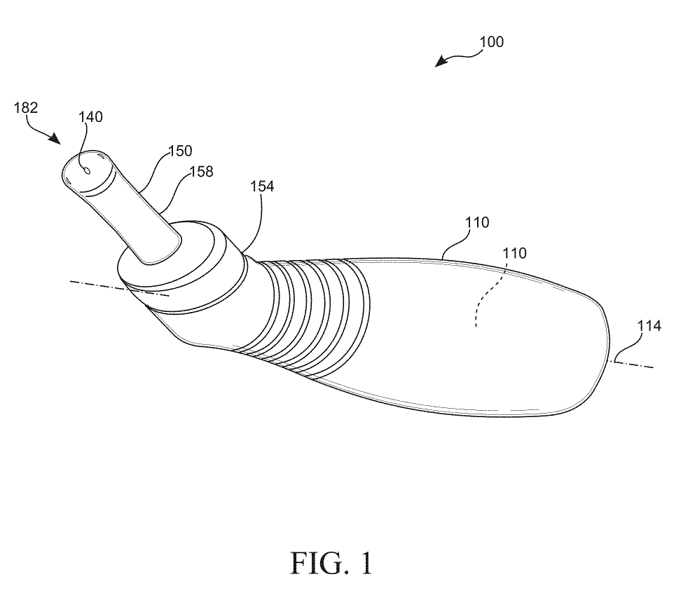

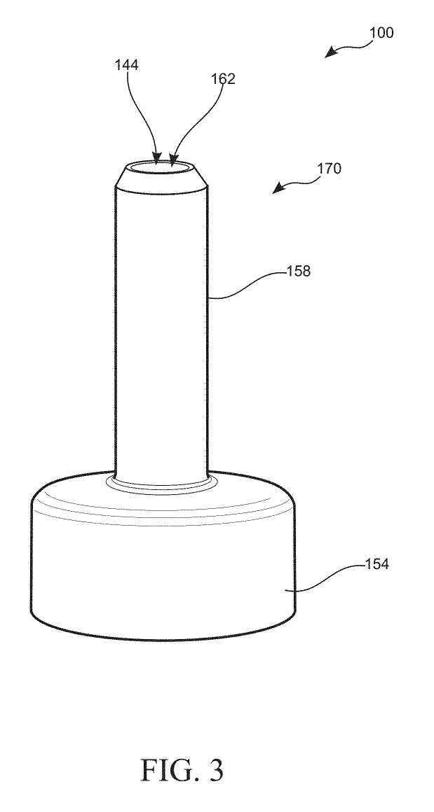

Referring to the drawings by numerals of reference there is shown in FIGS. 1-4, a perspective view illustrating personal hygiene bottle 100 according to an embodiment of the present invention.

Personal hygiene bottle 100 comprising: elongated container portion 110; flexible wall 130; aperture 140; first nozzle portion 150; and second nozzle portion 170. Elongated container portion 110 includes elongated interior volume 112 adapted to retain cleaning solution 120 therein. Cleaning solution 120 formed as water 124 and H₂O₂ solution 128. Cleaning solution 120 is placed within elongated interior volume 112 of elongated container portion 110.

Elongated container portion 110 forms elongated axis 114 as shown in FIG. 0.1. Elongated container portion 110 is formed from a material chosen from a list of materials consisting of rubber, plastic, and silicone.

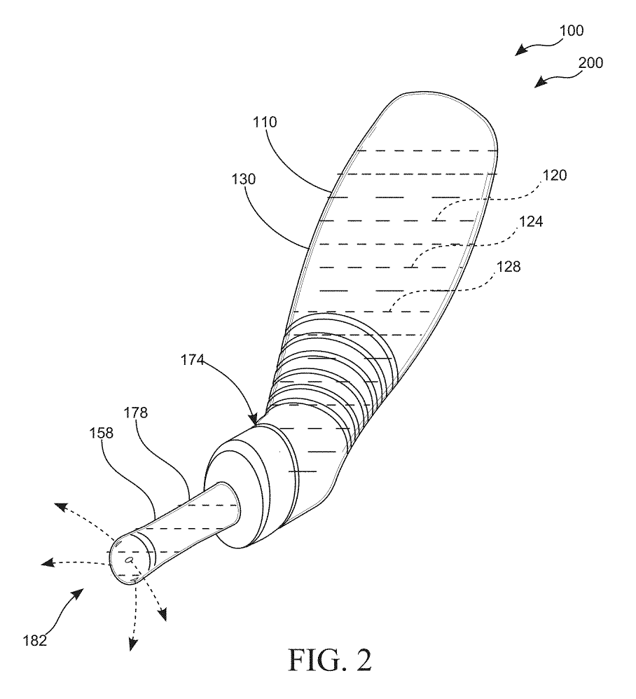

Flexible wall 130 is adapted such that when flexible wall 130 is squeezed said cleaning solution can be forced outwardly therefrom as shown in in-use condition 200 of FIG. 2. Flexible wall 130 is formed from a material chosen from a list of materials consisting of rubber, plastic, and silicone. Aperture 140 has center point 144 offset from axis 114 of elongated container portion 110 and is adapted to allow cleaning solution 120 to be forced outwardly from elongated container portion 110 as show in in in-use condition 200.

First nozzle portion 150 includes attachment portion 154, elongated neck portion 158, and single aperture 162. Attachment portion 154 is adapted to be releasably connected to elongated container portion 110 and surround aperture 162 thereof, such that cleaning solution 120 can be forced into nozzle portion 150. Elongated neck portion 158 adapted to allow cleaning solution 120 to pass therethrough. Single aperture 162 located on a distal end thereof adapted to allow cleaning solution 120 to flow outwardly therefrom such that cleaning solution 120 flows in a single stream at an angle to axis 114 of elongated container portion 110. First nozzle portion 150 is formed from a rigid plastic material.

Second nozzle portion 170 includes attachment portion 174, elongated neck portion 178, and plurality of spaced apertures 182 located on a distal end thereof. Attachment portion 174 adapted to be releasably connected to elongated container portion 110 and surround aperture 162 thereof, such that cleaning solution 120 can be forced into nozzle portion 170. Elongated neck portion 178 adapted to allow cleaning solution 120 to pass therethrough. Second nozzle portion 170 is formed from a rigid plastic material.

Plurality of spaced apertures 182 located on a distal end thereof each adapted to allow cleaning solution 120 to flow outwardly therefrom in a plurality of streams at differing angles to axis 114 of elongated container portion 110. User 210 can choose between first nozzle portion 150 and second nozzle portion 170 depending on the desired function of first bottle member 100.

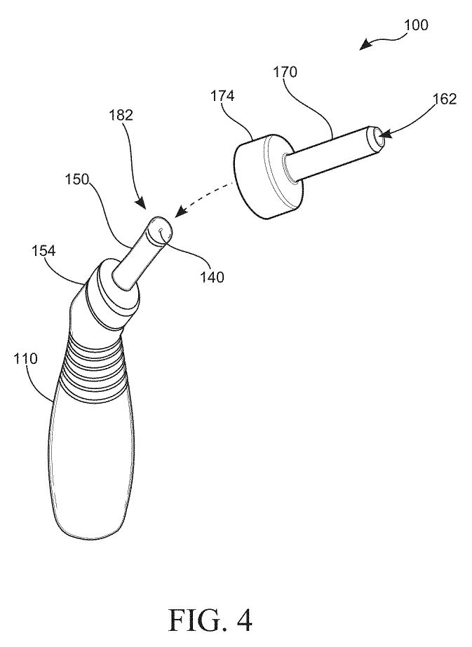

Referring now to FIGS. 4 & 5 showing perspective view illustrating personal hygiene kit 400 according to an embodiment of the present invention of FIG. 1

A personal hygiene kit 400 comprising: first bottle member 100; second bottle member 410; plurality of dry wipe members 440; mini-douche member 450; cleaning solution 120; and carrying pouch member 460. Second bottle member 410 includes elongated container portion 414 forming elongated axis 418, including elongated interior volume 420 adapted to retain cleaning solution 120 therein. Wherein elongated interior volume 420 of second bottle member 410 is larger than interior volume 112 of container portion 110 of first bottle member 100. Plurality of dry wipe members 440 are formed from a cotton material.

Cleaning solution 120 is adapted to be placed within the respective interior volumes 112 of first bottle member 100 and interior volume 420 of second bottle member 410. Carrying pouch member 460 is adapted to releasably retain first bottle member 100 and second bottle member 410, plurality of dry wipe members 440, mini-douche member 450, and cleaning solution 120 therein. Carrying pouch member 460 includes releasable connector member 464 adapted to releasably open and close an opening therein. Releasable connector member 464 may be formed as zipper member 466, female snap members, or hook and loop material.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. A personal hygiene bottle comprising:
an elongated container portion including:
an elongated interior volume adapted to retain a cleaning solution therein; and
wherein said elongated container portion forms an elongated axis;
a flexible wall;
wherein said flexible wall is adapted such that when said flexible wall is squeezed said cleaning solution can be forced outwardly therefrom; and
an aperture;
wherein said aperture has a center point offset from said axis of said elongated container portion and is adapted to allow said cleaning solution to be forced outwardly from said elongated container portion;
a first nozzle portion including:
an attachment portion adapted to be releasably connected to said elongated container portion and surround said aperture thereof, such that said cleaning solution can be forced into said nozzle portion;
an elongated neck portion adapted to allow said cleaning solution to pass therethrough; and
and a single aperture located on a distal end thereof adapted to allow said cleaning solution to flow outwardly therefrom such that said cleaning solution flows in a single stream at an angle to said axis of said elongated container portion; and
a second nozzle portion including:
an attachment portion adapted to be releasably connected to said elongated container portion and surround said aperture thereof, such that said cleaning solution can be forced into said nozzle portion; and
an elongated neck portion adapted to allow said cleaning solution to pass therethrough; and
and a plurality of spaced apertures located on a distal end thereof each adapted to allow said cleaning solution to flow outwardly therefrom in a plurality of streams at differing angles to said axis of said elongated container portion;
wherein a user can choose between said first and second nozzle portion depending on the desired function of said first bottle member.

2. The personal hygiene bottle of claim 1, further comprising a cleaning solution formed as a water and H₂O₂ solution; wherein said cleaning solution is placed within said elongated interior volume of said elongated container portion.

3. The personal hygiene bottle of claim 1, wherein said elongated container portion is formed from a material chosen from a list of materials consisting of rubber, plastic, and silicone.

4. The personal hygiene bottle of claim 1, wherein said flexible wall is formed from a material chosen from a list of materials consisting of rubber, plastic, and silicone.

5. The personal hygiene bottle of claim 1, wherein said first and second nozzle portions are formed from a rigid plastic material.

6. A personal hygiene kit comprising:
a first bottle member including:
a container portion including:
an interior volume adapted to retain a cleaning solution therein;
a flexible wall adapted such that when said flexible wall is squeezed said cleaning solution can be forced outwardly therefrom; and
an aperture adapted to allow said cleaning solution to be forced outwardly from said container portion;
a first nozzle portion including:
an attachment portion adapted to be releasably connected to said container portion and surround said aperture thereof, such that said cleaning solution can be forced into said nozzle portion;
an elongated neck portion adapted to allow said cleaning solution to pass therethrough; and
and a single aperture located on a distal end thereof adapted to allow said cleaning solution to flow outwardly therefrom such that said cleaning solution flows in a single stream; and
a second nozzle portion including:
an attachment portion adapted to be releasably connected to said container portion and surround said aperture thereof, such that said cleaning solution can be forced into said nozzle portion; and
an elongated neck portion adapted to allow said cleaning solution to pass therethrough; and
and a plurality of spaced apertures located on a distal end thereof each adapted to allow said cleaning solution to flow outwardly therefrom such that said cleaning solution flows in a a plurality of streams in different directions;
wherein a user can choose between said first and second nozzle portion depending on the desired function of said first bottle member;
a second bottle member including:
an elongated container portion forming an elongated axis, including:
an elongated interior volume adapted to retain a cleaning solution therein; wherein said elongated interior volume of said is larger than said interior volume of said container portion of said first bottle member;
a flexible wall adapted such that when said flexible wall is squeezed said cleaning solution can be forced outwardly therefrom; and
an aperture having a center point offset from said axis of said elongated container portion and adapted to allow said cleaning solution to be forced outwardly from said container portion;
a first nozzle portion including:
an attachment portion adapted to be releasably connected to said elongated container portion and surround said aperture thereof, such that said cleaning solution can be forced into said nozzle portion;
an elongated neck portion adapted to allow said cleaning solution to pass therethrough; and
and a single aperture located on a distal end thereof adapted to allow said cleaning solution to flow outwardly therefrom such that said cleaning solution flows in a single stream at an angle to said axis of said elongated container portion; and
a second nozzle portion including:
an attachment portion adapted to be releasably connected to said elongated portion and surround said aperture thereof, such that said cleaning solution can be forced into said nozzle portion; and
an elongated neck portion adapted to allow said cleaning solution to pass therethrough; and
and a plurality of spaced apertures located on a distal end thereof each adapted to allow said cleaning solution to flow outwardly therefrom in a plurality of streams at differing angles to said axis of said elongated container portion;
wherein a user can choose between said first and second nozzle portion depending on the desired function of said second bottle member;
a plurality of dry wipe members;
a mini-douche member; and
a cleaning solution;
wherein said cleaning solution is adapted to be placed within respective said interior volumes of said first and second bottle members; and
a carrying pouch member;
wherein said carrying pouch member is adapted to releasably retain said first and second bottle members, said plurality of dry wipe members, said mini-douche member, and said cleaning solution therein.

7. The personal hygiene bottle of claim 6, further comprising a cleaning solution formed as a water and H₂O₂ solution; wherein said cleaning solution is placed within said elongated interior volume of said elongated container portion.

8. The personal hygiene bottle of claim 6, wherein said elongated container portion is formed from a material chosen from a list of materials consisting of rubber, plastic, and silicone.

9. The personal hygiene bottle of claim 6, wherein said flexible wall is formed from a material chosen from a list of materials consisting of rubber, plastic, and silicone.

10. The personal hygiene bottle of claim 6, wherein said first and second nozzle portions are formed from a rigid plastic material.

11. The personal hygiene kit of claim 6, wherein said plurality of dry wipe members are formed from a cotton material.

12. The personal hygiene kit of claim 6, wherein said carrying pouch member includes a releasable connector member adapted to releasably open and close an opening therein.

13. The personal hygiene kit of claim 12, wherein said releasable connector member is formed as a zipper member.

14. The personal hygiene kit of claim 12, wherein said releasable connector member is formed from male and female snap members.

15. The personal hygiene kit of claim 12, wherein said releasable connector member is formed from hook and loop material.
